(19) United States
(12) Patent Application Publication
(10) Pub. No.: US 2026/0232255 A1
(43) Pub. Date: Aug. 13, 2026

(54) INPUT ATTACHMENT, SURGICAL DEVICE, ARRANGEMENT FOR INTRAOPERATIVE USE AND USE OF AN INPUT ATTACHMENT

(71) Applicant: inomed Medizintechnik GmbH, Emmendingen (DE)

(72) Inventors: Georg Hauser, Offenburg (DE); Damir Pfau, Umkirch (DE); Kornelius Lente, Lahr (DE)

(21) Appl. No.: 19/121,720

(22) PCT Filed: Oct. 10, 2023
(86) PCT No.: PCT/EP2023/077954

§ 371 Date: Apr. 16, 2025

(30) Foreign Application Priority Data
Oct. 18, 2022 (DE) 10 2022 211 015.3

(51) Int. Cl. A61B 5/388; A61B 5/00

(52) U.S. Cl. CPC A61B 5/388; A61B 5/0004; A61B 5/7405; A61B 5/742; A61B 5/7455; A61B 5/7475; A61B 2217/005; A61B 2505/05; A61B 2560/045

(57) ABSTRACT

Input attachment, surgical device, arrangement for intraoperative use and use of the input attachment The present invention relates to an input attachment for controlling at least one function of a sterile surgical handpiece via an external surgical device coupled therewith, wherein the input attachment comprises: an input device which is configured to receive a user input; a control device which is configured to generate a control signal for controlling the surgical handpiece from the user input received, an electrical interface through which the control signal generated is transmitted to the surgical device, and a mechanical assembly interface which is configured to form a non-slip connection with the surgical handpiece in the assembled state of the input attachment in such a way that the input attachment assembled with the surgical handpiece can be handled as a one-piece handpiece. Besides, the invention relates to a surgical device, an arrangement for intraoperative use and use of the input attachment for interoperative neuromonitoring (IOM) according to the invention.

### FIELD OF INVENTION

The present invention relates to an input attachment for controlling at least one function of a sterile surgical handpiece via an external surgical device coupled therewith, a surgical device, an arrangement for intraoperative use and a use of a corresponding input attachment.

### TECHNICAL BACKGROUND

For intraoperative neuromonitoring (IOM), surgical handpieces such as a stimulation probe or a mapping suction device are used during surgery, with which a surgeon can stimulate a nerve with a small electrical impulse. For this purpose, the handpiece must be sterile in order not to cause any contamination at the surgical site. For this purpose, it is advantageous to have an adjustable current intensity for stimulating the nerves, while also having an indicator for the stimulation response. It is often the case that the smaller the current intensity required to trigger a signal, the closer you are to the area to be protected. Adjusting the current intensity is often only achieved by connecting an external device to the handpiece. The EP 1 804 911 B1, on the other hand, describes a stimulator handpiece comprising a switch that can be used to adjust an electrical signal.

### SUMMARY OF THE INVENTION

In the light of the above background, it is an object of the present invention to provide simple control option for the surgeon for a large number of different surgical handpieces.

According to the present invention, this object is achieved by an input attachment having the features of patent claim 1, a surgical device having the features of claim 25, an arrangement having the features of patent claim 27 and/or a use of the input attachment having the features of patent claim 31.

Accordingly, the present invention provides
An input attachment for controlling at least one function of a sterile surgical handpiece via an external surgical device coupled therewith, wherein the input attachment comprises: an input device which is configured to receive a user input; a control device which is configured to generate a control signal for controlling the surgical handpiece from the user input received, an electrical interface through which the control signal generated is transmitted to the surgical device, and a mechanical assembly interface which is configured to form a non-slip connection with the surgical handpiece in the assembled state of the input attachment in such a way that the input attachment assembled with the surgical handpiece can be handled as a one-piece handpiece.
A surgical device, comprising a first electrical device interface having a first interface and a second interface, wherein the first interface is configured to receive control signals from an input attachment coupled to the surgical device via the electrical device interface according to the invention, and wherein the second interface is configured to transmit stimulation signals based on the control signals received to a surgical handpiece coupled to the surgical device via the electrical device interface.
An arrangement for intraoperative use, comprising: an input attachment according to the invention, a surgical device according to the invention, and a surgical handpiece.
Use of the input attachment according to the invention for interoperative neuromonitoring (IOM).

The idea underlying the present invention is to develop an attachment having an input function, said attachment being capable of being easily placed on and attached to a handpiece of a surgical instrument or surgical handpiece. This makes it possible for the surgeon to adjust parameters such as current intensity during the operation without moving away from or turning away from the surgical site. Furthermore, the same attachment can be mounted on another handpiece if required, in order to adjust the parameters there in the same manner.

With the attachment in place, the surgeon can easily control the surgical instrument or surgical handpiece from the sterile area. This is important since the surgical device connected is often not sterile and the surgeon would have to turn away from the operating table.

Thus, the input attachment can be used for different surgical instruments. Included in this are various probes for, for example, the brain surface or deeper brain areas, HF devices or scalpels. For this, the input attachment should be mounted on the instrument that the surgeon changes the least during the operation.

In this manner, the surgical handpiece, such as a stimulation probe or a mapping suction device, can be more easily configured to be used for simple procedures without a controller. Since the input attachment is comprised of a lower risk class, the development of a high-risk probe is not necessary. The input attachment can also be implemented as a disposable product. This also eliminates the need for any time-consuming reprocessing of the attachment.

The input attachment records inputs from the treating surgeon, converts them into a control signal and transmits them to a connected surgical device. To do this, it uses an electrical interface that is configured to transmit electrical signals. The inputs can be made, for example, using one or more buttons or keys, a control dial or a slide control.

The medical surgical device belonging to the input attachment has the appropriate interfaces to convert the control signal transmitted by the input attachment into a stimulation signal or another output, which is then transmitted to the surgical handpiece coupled to the surgical device.

Thus, an arrangement is formed from the three components, namely the input attachment, the surgical device and the surgical handpiece. This arrangement can then be used in various surgical fields, such as in intraoperative neuromonitoring. This use includes, for example, motor mapping and speech mapping in neurosurgery, as well as applications in spinal surgery and peripheral surgery, particularly for tumor resection.

In motor mapping, for example, tissue is stimulated to locate structures in the brain that control movement. If stimulation-evoked potentials can be displayed in the characteristic muscles, a function to be protected must be located near the stimulated area. These include, for example, subcortical mapping according to Raabe for tumor resection in neurosurgery or mapping of primary motor cortex and pyramidal tracts, whereby the latter require special probe shapes and parameters.

The probes used are mainly monopolar probes with ball tips, fork probes, mapping suction devices, small bipolar concentric probes (BCS) or micro-fork probes.

Tumor surgery may be performed in ENT surgery, general surgery, visceral surgery, endocrine surgery or oral and maxillofacial surgery. The current intensity is mainly used as the parameter to be controlled. For example, the invention is used for tumors on the facial nerve, the thyroid gland or rectal cancer. Here, the nerve can be distinguished from the surrounding tumor tissue on the basis of its lower current threshold for triggering a response signal.

A one-piece handpiece is a medical tool or instrument to be held in one hand, which is usually usable by a surgeon during surgery. Even if this comprises at least two components, namely a surgical handpiece with an attached input attachment, the one-piece handpiece does not differ in its handling from an instrument consisting of only one component due to the stable and non-slip attachment of the components.

A non-slip connection means that the two components of the connection, in this case the surgical handpiece and the input attachment, are attached to each other in such a way that the relative position of the two components does not change, even with only a slight force applied during normal use of the handpiece. This means that as long as no excessive force is used, which would not occur during normal use, the two components will not slip relative to each other.

Advantageous embodiments and further configurations result from the dependent claims as well as from the description with reference to the figures of the drawing.

According to a preferred further development, the assembly interface comprises a receiving region. The receiving region is configured to form a form-fitting and/or force-fitting connection between the input attachment and the surgical handpiece when the input attachment is placed on the surgical handpiece. In this manner, a non-slip handpiece that can be handled in one piece is made possible between the input attachment and the surgical handpiece.

according to a preferred further development, the form-fitting and/or force-fitting connection comprises at least one of the following connections: At least one clip for clipping the receiving region to the surgical handpiece, at least one magnet for magnetic coupling to an opposite pole magnet on the surgical handpiece, an adhesive connection between the receiving region and the surgical handpiece, a bonding connection for bonding the receiving region to the surgical handpiece; or a form-fitting dovetail connection or dovetail-like form-fitting connection to the surgical handpiece. Each of these connections provide a firm, non-slip and solid one-piece handpiece for use in surgery.

According to a further preferred further development, the form-fitting and/or force-fitting connection can be released again. This enables the input attachment and the surgical handpiece to be replaced and reused. The interchangeability of the input attachment makes it possible to attach the attachment to other surgical handpieces without having to use another attachment.

According to a further preferred further development, the receiving region of the assembly interface is configured to insert the surgical handpiece into an inner region of the receiving region in a non-assembled state and to enclose it in the receiving region, and to clamp the surgical handpiece in the assembled state in the inner region of the receiving region so that it cannot slip. This further development also enables a fixed and solid one-piece handpiece, in particular for attachment to a larger device such as a mapping suction device.

According to another further development, the non-slip connection is configured to use anti-slip elements provided in the receiving region. The anti-slip elements make it more difficult and in particular prevent the input attachment from slipping off the surgical handpiece when assembled.

According to a further preferred further development, the non-slip connection is configured by protuberances provided in the receiving region, which in the assembled state engage in inlets of the surgical handpiece and at least make it more difficult and in particular prevent the input attachment from slipping off the surgical handpiece. The protuberances are configured to be sufficiently deformable to enable engagement in the inlets on the one hand and to make it more difficult for the attached input attachment to slip or to prevent it from slipping on the other.

According to another preferred further development, the electrical interface comprises a bipolar electrical connection. The input signal to the surgical device can be received directly via this connection, and a response signal from the surgical device can also be received. The response signal is therefore usually a response, for example a stimulus response of the stimulation or output from the surgical handpiece to the site to be treated, for example in the brain tissue. Such a connection enables fast and latency-free transmission of signals.

According to another preferred further development, the electrical interface is configured to couple the surgical handpiece to the input attachment via a direct cable connection. The cable connection enables, for example, secure and stable data exchange with the surgical device.

According to a preferred further development, the electrical interface is configured to wirelessly couple the surgical handpiece with the input attachment via an air interface. This connection enables increased freedom of movement for persons involved, such as the surgeon, without having to pay attention to another cable.

According to another preferred further development, the air interface is an optical connection. The optical connection can be configured to be an infrared connection. The connection can also be configured to be a radio connection, for example a WLAN or mobile radio connection and/or a Bluetooth connection. Depending on the requirements of the specific application, these connections enable sufficiently secure and fast data transmission.

According to another preferred further development, the electrical interface is arranged in the area of the mechanical assembly interface. Furthermore, the mechanical assembly interface is configured to be coupled in the assembled state with a corresponding electrical interface of the surgical handpiece in order to transmit the input signal to the surgical device via the surgical handpiece in this manner and to receive a response signal from the surgical device. In this manner, signals from or to the surgical handpiece and the input attachment can be sent and received via the same connection, for example a cable connection with only one cable. This simplifies the complexity of the arrangement.

According to another further development, the input device comprises at least one button, a keyboard, a touchpad, a rotary knob and/or a scroll wheel, via which user input can be recorded. This allows the surgeon to control the surgical handpiece in a simple and flexible manner.

According to another further development, the input device is configured to output haptic feedback via user input. In this manner, for example, a haptic signal can be output when the surgeon has successfully made an input, so that the surgeon knows that the input has been received. This increases the safety of using the handpiece.

According to another further development, the input device and the control device are configured to record an input parameter for stimulation in the field of interoperative neuromonitoring as user input and convert it into a corresponding control signal. In this case, the parameter is in particular a current intensity or a frequency, which are parameters that are frequently varied. However, the parameter can also be a pulse width, an energy, a stimulation frequency, for example 1 Hz or 30 Hz, a pulse shape or switching from a monopolar to a bipolar signal, or simply switching the stimulation on and off. The latter is preferred when using a mapping suction device. In this manner, the surgical handpiece can be used effectively in intraoperative neuromonitoring.

According to another further development, the input device and the control device are configured to select at least one parameter of a parameter set, to receive the selected parameter as the user input and to convert it into a corresponding control signal. A parameter set can also be switched automatically depending on a completed step in the workflow. In this manner, the surgical handpiece can be used in a particularly flexible and user-friendly manner in intraoperative neuromonitoring (IOM).

According to another further development, the control device is configured to use the control signal to control at least one device-specific parameter of the surgical handpiece or the surgical device. These include, in particular, a comment, a workflow, a volume, a baseline, switching back and forth in the workflow, opening a comment menu with subsequent setting of a standardized comment, or other parameters relevant to the working environment. In this way, an improved working environment can be created for the surgeon, which reduces treatment errors.

According to a further preferred further development, a display device is provided which is configured to output a response signal. The response signal is received by the surgical handpiece and sent to the input attachment directly or indirectly, for example via the surgical device, where it is converted into the corresponding response signal. Such a response signal can, in particular, be an optical response signal. This is clearly visible to the surgeon so that he or she quickly notices the reaction of the stimulated nerve, for example, and quickly adjusts the treatment accordingly.

According to another further development, the display device is configured to display a reaction signal received which is based on a reaction to a stimulation or output issued by the surgical handpiece and based on the user input. The response signal thus provides information about the response generated by the control signal in, for example, the stimulated area of a patient.

According to another further development, the display device comprises a display and/or at least one LED for outputting the response signal. The LED can be configured to be an RGB LED for displaying different colors, in particular according to a traffic light scheme. Furthermore, the LED can be designed for flashing and static lighting. In particular, the display can be configured to be a mini display, optionally with haptic feedback. This type of display is particularly easy to recognize and flexible to use.

According to another further development, the display device is configured to display at least one parameter selected and/or a parameter value and/or a parameter range and/or an optical warning signal. In this way, useful information can be provided for the surgeon.

According to another further development, the display device is configured to display at least one parameter of the surgical handpiece, the surgical device and/or a third-party device connected to the input attachment. In the case of navigating a probe of the surgical handpiece with a corresponding device, the response could be displayed in relation to the current location. In this manner, the surgeon can recognize the magnitude of the parameter currently in use and proceed with the treatment accordingly. In this way, the surgeon can recognize suitable parameter ranges, which increases the safety and precision of the treatment.

According to another further development, the display device is designed to output haptic feedback and/or an acoustic response signal. In this manner, special attention can be generated for the treating surgeon. This can be used, for example, if parameter ranges are exceeded. Overall, this increases the parameter setting as well as the safety during treatment.

According to another further development, the display device is coupled to the control device, via which the display device can be controlled. In this manner, the display of various response signals can be set flexibly and individually for the surgeon, which improves the applicability and usability of the input attachment.

According to a further development, the surgical device is configured to be an interoperative neuromonitoring (IOM) device. This enables neurological applications, such as the stimulation of tissue in the brain, for example to carry out mapping in the brain to locate areas in the brain that control speech or motor function. Alternatively, the surgical device can also be an RF or cryotherapy device.

According to a further development, the surgical device is electrically connected to the input attachment and the surgical handpiece for transmitting and receiving signals. Thus, inputs made via the input attachment can first be transmitted to the surgical device as a control signal, and from this, for example, a corresponding stimulation signal can be trans-mitted to the surgical handpiece.

Furthermore, the surgical handpiece can be mechanically attached to the input attachment in such a way that the input attachment is attached to the position of the surgical handpiece corresponding to the receiving region of the input attachment. The one-piece handpiece assembled in this manner is then particularly easy to handle, which is essential when treating a patient.

According to a further development, the surgical handpiece is configured to be a monopolar or bipolar stimulation probe. According to another further development, the surgical handpiece is configured to be a monopolar or bipolar mapping suction device. These devices enable more effective intraoperative neuromonitoring.

The above embodiments and modifications may be combined with each other in any sensible way. In particular, all features of the input attachment and the surgical device are transferable to the arrangement and all features of the input attachment are transferable to the use of the input attachment for interoperative neuromonitoring, and vice versa. Further possible embodiments, further configurations and implementations of the invention also comprise combinations, not explicitly mentioned, of features of the invention described above or below with respect to the exemplary embodiment. In this respect, the skilled person will in particular also add individual aspects as improvements or additions to the respective basic form of the present invention.

### DESCRIPTION OF THE DRAWINGS

The present invention will be explained in more detail below using the examples given in the schematic figures of the drawing, wherein: wherein:

FIG. 1 shows a schematic diagram of an arrangement of an input attachment, a surgical handpiece and a surgical device according to a first embodiment;

FIG. 2 shows a schematic diagram of an input attachment of a further embodiment;

FIG. 3 shows a cross-section of the input attachment from FIG. 2;

FIG. 4 shows a schematic diagram of a surgical handpiece with the input attachment of FIGS. 2 and 3 in place;

FIG. 5 shows a schematic diagram of an input attachment of a further embodiment;

FIG. 6 shows a cross-section of the input attachment o a further embodiment;

FIG. 7 is a schematic diagram of a surgical handpiece with the input attachment of FIG. 5 or 6 in place;

FIG. 8 shows a schematic diagram of an arrangement comprising an input attachment, surgical handpiece and surgical device according to a further embodiment;

FIG. 9 shows a schematic diagram of an arrangement comprising an input attachment, surgical handpiece and surgical device according to a further embodiment ;

FIG. 10 shows a schematic diagram of an arrangement comprising an input attachment, surgical handpiece and surgical device according to a further embodiment;

FIG. 11 shows a schematic diagram of an arrangement comprising an input attachment, surgical handpiece and surgical device according to a further embodiment; and

FIG. 12 shows a schematic diagram of an arrangement comprising an input attachment, surgical handpiece and surgical device according to a further embodiment.

The accompanying figures are intended to convey a further understanding of the embodiments of the invention. They illustrate embodiments and, in connection with the description, serve to explain principles and concepts of the invention. Other embodiments and many of the advantages mentioned will be apparent with reference to the drawings. The elements shown in the drawings are not necessarily shown to scale with respect to each other.

In the figures of the drawing, the same elements, features and components, which have the same function and act in the same way, are each identified with the same reference signs, unless otherwise stated.

### DESCRIPTION OF EXEMPLARY EMBODIMENTS

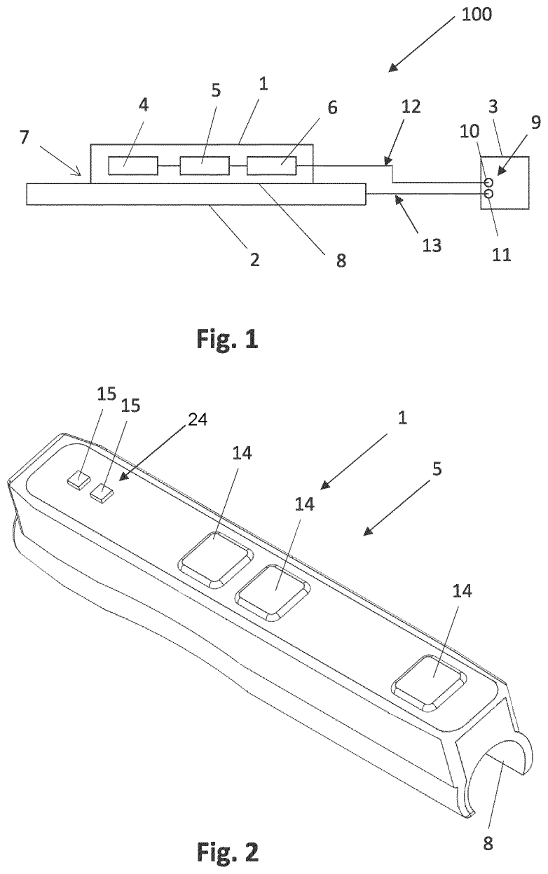

FIG. 1 shows a schematic diagram of an arrangement 100 comprising input attachment 1, surgical handpiece 2 and surgical device 3 according to a first embodiment;

FIG. 1 shows an arrangement 100 for intraoperative use. The arrangement comprises an input attachment 1, a surgical device 3 electrically connected thereto, and a surgical handpiece 2 connected to the surgical device 3. During an operation, the input attachment 1 and the surgical handpiece are sterile. The input attachment 1 is configured to control at least one function of the surgical handpiece 2 via the surgical device 3.

The input attachment 1 comprises an input device 4, for example buttons or a rotary control. The input device 4 is configured to receive user input and transmit it to a control device 5 of the input attachment 1. The control device 5 is configured to generate a control signal for controlling the surgical handpiece 2 from the user input received. This is sent to an electrical interface 6, via which the control signal is transmitted to the surgical device 3.

The electrical interface 6 comprises a monopolar electrical connection via which the input signal can be transmitted to the surgical device 3. In this embodiment, this is implemented by a first connection 12 configured to be a cable connection.

The input attachment 1 also comprises a mechanical assembly interface 7. This is configured to form a non-slip connection with the surgical handpiece 2 in the assembled state of the input attachment 1 in such a manner that the input attachment 1 assembled with the surgical handpiece 2 can be handled as a one-piece handpiece.

The assembly interface 7 comprises a receiving region 8 which is configured to form a form-fitting and/or force-fitting connection between the input attachment 1 and the surgical handpiece 2 when the input attachment 1 is placed on the surgical handpiece 2.

In this embodiment, the mechanical connection is configured to be a form-fitting connection and comprises at least one clip for clipping the receiving region 8 to the surgical handpiece 2. In further embodiments, the form-fitting and/or force-fitting connection comprises at least one magnet for magnetic coupling to an opposite pole magnet on the surgical handpiece 2, an adhesive connection between the receiving region 8 and the surgical handpiece 2, a bonding connection for bonding the receiving region 8 to the surgical handpiece 2, a form-fitting dovetail connection or dovetail-like form-fitting connection to the surgical handpiece 2.

In particular, the form-fitting and/or force-fitting connection is configured to be detachable.

The medical surgical device 3 shown in FIG. 1 has an electrical device interface 9 comprising a first 10 and a second interface 11.

The first interface 10 is configured to receive control signals from an input attachment 1 coupled to the surgical device 3 via the electrical device interface 9. The second interface 11 is configured to transmit stimulation signals based on the received control signals to a surgical handpiece 2 coupled to the surgical device 3 via the electrical device interface 9. This is achieved via a second electrical connection 13, which is configured to be a cable connection in this embodiment. Thus, it can be achieved that the input entered by the surgeon at the input attachment is converted into a stimulation or an output in the surgical handpiece and output.

The input device 4 and the control device 5 are configured to receive an input parameter for stimulation in the field of interoperative neuromonitoring as user input and to convert it into a corresponding control signal. In particular, the parameter is a current intensity. In further embodiments, the parameter is a frequency, a pulse width, a pulse shape, a voltage, an energy, or an on/off switching of a function.

The input device 4 and the control device 5 are also configured to select at least one parameter of a parameter set, to receive the selected parameter as the user input and to convert it into a corresponding control signal. Thus, it can be switched back and forth between different parameters, for example between a current intensity or a frequency.

FIG. 2 shows a schematic diagram of an input attachment 1 of a further embodiment;

In this embodiment, the input device 4 comprises at least one button 14 via which user input can be received. In further embodiments, the input device comprises a keyboard, a touch-pad, a rotary knob and/or a scroll wheel. Furthermore, the input device 4 is configured to output haptic feedback via user input. This is done here by vibrating the button 14 during input.

The receiving region 8 of the mechanical interface 7 is configured here as a rounding for a cylindrical surgical handpiece 2.

In this embodiment, the input attachment also comprises a display device 24, which is configured to output a response signal. The response signal output here is output via two LEDs 15 as an optical response signal.

The electrical interface 6 not shown in FIG. 2 is configured to receive the response signal from the surgical device 3. The display device 24 is configured to display the received response signal. This signal is received and displayed here as a reaction to a stimulation or output issued by the surgical handpiece 2 and based on the user input.

The display device 24 is coupled to the control device 5, and is controllable or adjustable via the control device 5, which is also not shown here.

In further embodiments, the display device 24 comprises a display.

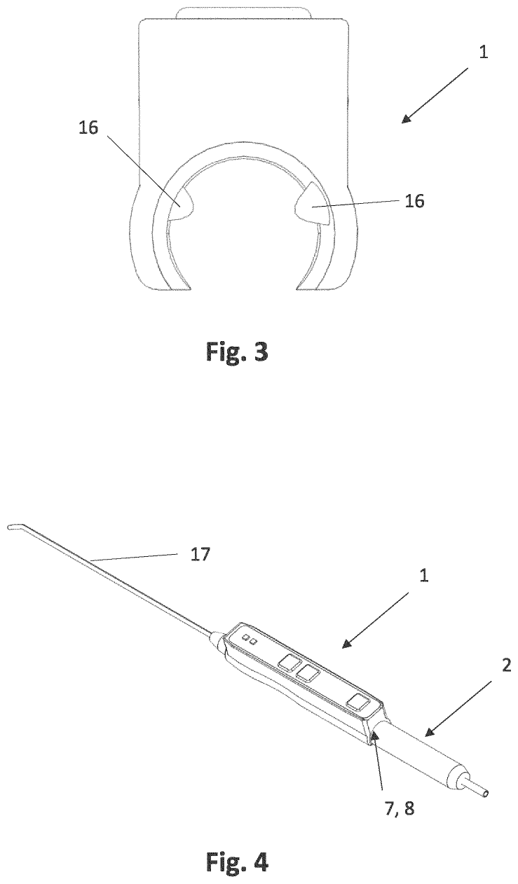

FIG. 3 shows a cross-section of the input attachment 1 of FIG. 2.

What can be seen are engaging protuberances 16 for engaging in corresponding recesses on the outside of the surgical handpiece 2. Thus, between the input attachment 1 and the surgical handpiece 2 there is established a secure, non-slip and detachable mechanical connection.

FIG. 4 shows a schematic diagram of a surgical handpiece 2 with the input attachment of FIGS. 2 and 3 in place.

In this embodiment, the surgical handpiece 3 is configured to be a monopolar stimulation probe. In further embodiments, the surgical handpiece 3 is configured to be a bipolar stimulation probe. The surgical handpiece therefore comprises a probe 17 that is angled at its tip. In further embodiments, the probe is angled at different angles. Shorter or longer probes are also possible, depending on the area of application.

The input attachment 1 is used for interoperative neuromonitoring (IOM). The assembled state of the input attachment with the surgical handpiece 2 can be clearly seen in FIG. 4. In particular, it can be seen that the mechanical assembly interface 7 is configured to form a non-slip connection with the surgical handpiece 2 in the assembled state of the input attachment such that the input attachment 1 assembled with the surgical handpiece 2 can be handled as an easily and practically usable one-piece handpiece.

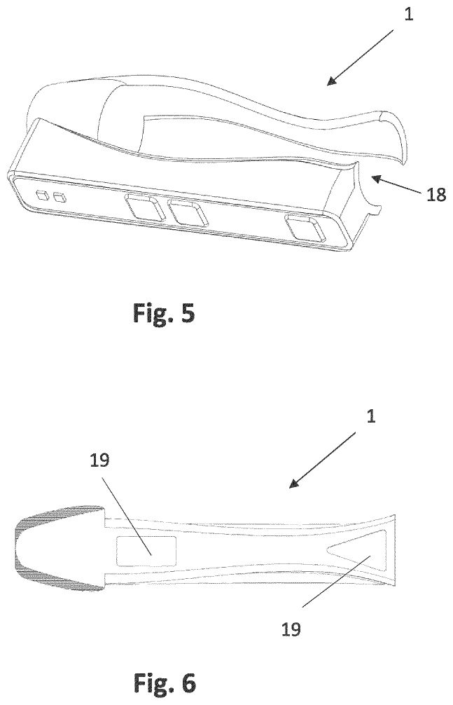

FIG. 5 shows a schematic diagram of an input attachment 1 of a further embodiment;

In this further embodiment of the input attachment 1, a receiving region 8 of the assembly interface 7 is configured to insert the surgical handpiece 2 into an inner region 18 of the receiving region 8 in a non-assembled state and to enclose it in the receiving region 8, and to clamp the surgical handpiece 2 in the assembled state in the inner region 18 of the receiving region 8 so that it cannot slip.

FIG. 6 shows a cross-section of an input attachment of a further embodiment.

In this embodiment of the input attachment 1, the non-slip connection is configured by anti-slip elements 19 provided in the receiving region 8, which at least make it more difficult for the input attachment 1 to slip off the surgical handpiece 2 in the assembled state and practically prevent it in normal use. The anti-slip elements 19 can be configured, for example, from a plastic material. Here, the anti-slip elements 19 are implemented as inlets for the surgical handpiece 2.

In further embodiments, the non-slip connection can be configured to include protuberances provided in the receiving region which, in the assembled state, engage in inlets of the surgical handpiece 2 and at least make it more difficult for the input attachment to slip off the surgical handpiece 2 and, in particular, prevent it from doing so.

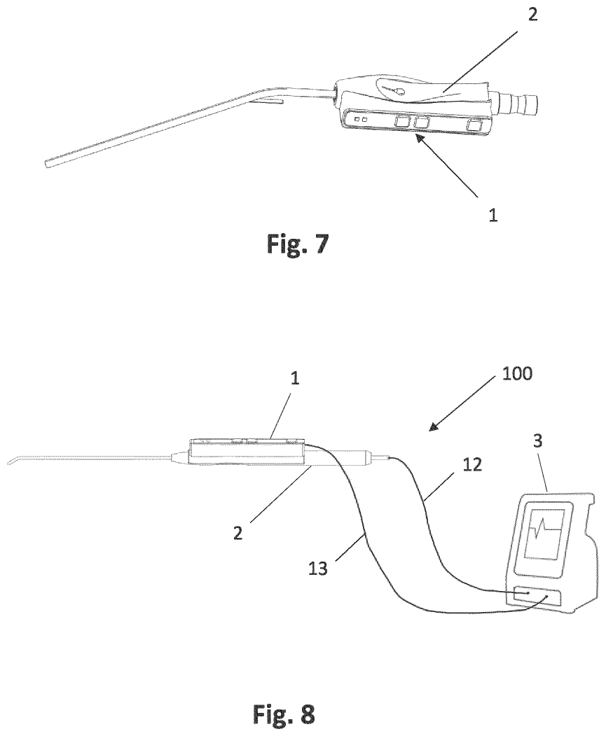

FIG. 7 shows a schematic diagram of a surgical handpiece 2 with the input attachment 1 of FIG. 5 or 6 in place.

In this embodiment, the surgical handpiece 2 is configured to be a monopolar mapping suction device. In further embodiments, the surgical handpiece 2 is configured to be a bipolar mapping suction device. Thus, in this embodiment, the input attachment 1 is used for interoperative neuromonitoring (IOM).

Analogous to FIG. 4, in which the input attachment 1 is configured to be used with a stimulation probe, the assembled state of the input attachment 1 configured for the mapping suction device with the surgical handpiece 2 can be clearly seen here. It can thus be seen that the input attachment 1 assembled with the surgical handpiece 2 can be handled as a practically usable one-piece handpiece.

FIG. 8 shows a schematic diagram of an arrangement 100 comprising input attachment 1, surgical handpiece 2 and surgical device 3 according to a further embodiment;

The input attachment 1 and the surgical handpiece are connected to the surgical device 3, which is configured to be an interoperative neuromonitoring (IOM) device.

The surgical device 3 is electrically connected to the input attachment 1 and the surgical handpiece 2 for transmitting and receiving signals via the first and second electrical connections 12, 13, both of which are configured here as cable connections. The surgical handpiece 2 is mechanically attached to the input attachment 1 in such a way that the input attachment 1 is attached to a position of the surgical handpiece 2 corresponding to the receiving region 8 of the input attachment 1. Thus, there is formed a handpiece that is easy to handle for the surgeon.

In this embodiment, the control device 5 is also configured to use the control signal to control at least one device-specific parameter of the surgical handpiece 2 or the surgical device 3. For example, here there can be controlled a comment or a workflow of the surgical device 3. For example, comments can be set for thyroid operations, such as “start surgery”, “before resection left”, “after resection left”, or a workflow or a wizard or device navigation can be switched back and forth between different action steps.

In further embodiments, the electrical interface 6 is configured to couple the surgical handpiece 2 to the input attachment 1 via a direct cable connection.

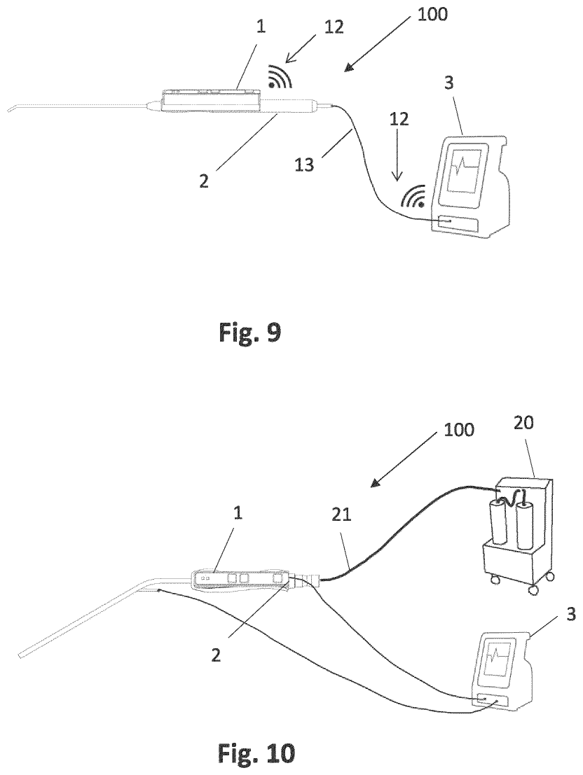

FIG. 9 shows a schematic diagram of an arrangement 100 comprising input attachment 1, surgical handpiece 2 and surgical device 3 according to a further embodiment;

In this embodiment, the electrical interface 6 is configured to wirelessly couple the surgical handpiece 2 to the input attachment via an air interface.

The air interface is configured to be a radio connection in the form of a Bluetooth connection. In further embodiments, the air interface is configured to be an optical connection, in particular an infrared connection. In further embodiments, the radio connection is configured to be a WLAN or mobile radio connection.

FIG. 10 shows a schematic diagram of an arrangement 100 comprising input attachment 1, surgical handpiece 2 and surgical device 3 according to a further embodiment;

In this arrangement 100, the surgical handpiece 3 is configured to be a mapping suction device. A surgical suction device 20 is connected to the surgical handpiece 2 in order to suction off tissue via a tube 21, for example during an operation.

The display device 24 is configured to display at least one parameter of a third-party device connected to the input attachment 1, such as the surgical suction device in this case.

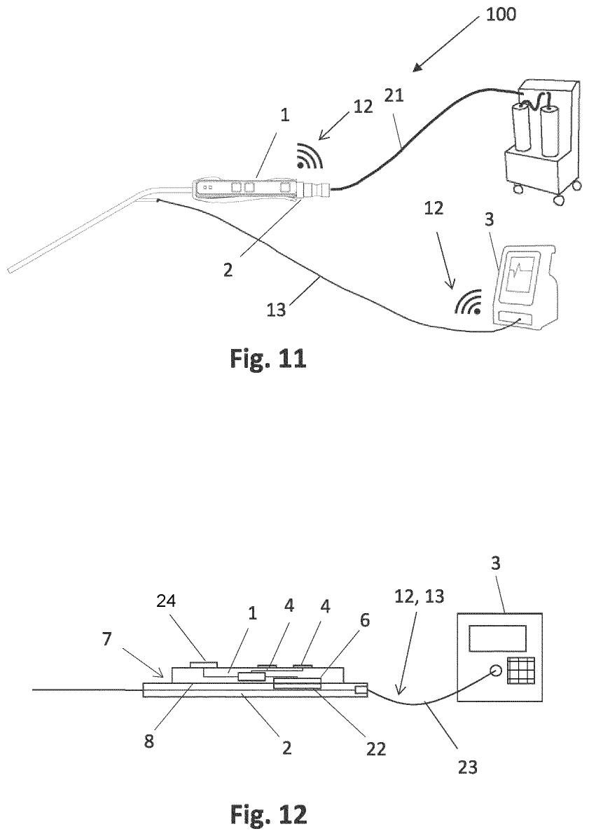

FIG. 11 shows a schematic diagram of an arrangement comprising input attachment 1, surgical handpiece 2 and surgical device 3 according to a further embodiment;

In this embodiment, the first electrical connection 12 is again configured to be a Bluetooth connection. Further embodiments use other wireless connections, as already described above in FIG. 9.

FIG. 12 shows a schematic diagram of an arrangement 100 comprising input attachment 1, surgical handpiece 2 and surgical device 3 according to a further embodiment;

In this embodiment, the electrical interface 6 of the input attachment 1 is arranged in the area of the mechanical interface. The electrical interface 6 is configured to be coupled in the assembled state to a corresponding electrical interface 22 of the surgical handpiece 2, in this manner transmit-ting the input signal to the surgical device 3 via the surgical handpiece 2 and receiving a response signal from the surgical device 3. Thus, the communication of the first and second electrical connection 12, 13 between the surgical handpiece 2 and input attachment 1 on the one hand and the surgical device 3 on the other hand takes place via a common cable 23.

Although the present invention has been fully described above on the basis of preferred exemplary embodiments, the present invention is not limited thereto, but may be modified in a variety of ways.

### LIST OF REFERENCE SIGNS

1 input attachment
2 surgical handpiece
3 surgical device
4 input device
5 control device
6 electrical interface of the input attachment
7 assembly interface
8 receiving region
9 device interface of the surgical device
10 first electrical interface of the surgical device
11 second electrical interface of the surgical device
12 first electrical connection
13 second electrical connection
14 button
15 LED
16 engaging protuberances/protuberances
17 probe
18 inner region of the input attachment
19 anti-slip element
20 surgical suction device
21 tube
22 receiving region of the surgical handpiece
23 common cable
24 display device
100 arrangement

1. An input attachment (1) for controlling at least one function of a sterile surgical handpiece (2) via an external surgical device (3) coupled therewith, the input attachment (1) comprising:
an input device (4) which is configured to receive a user input;
a control device (5) which is configured to generate a control signal for controlling the surgical handpiece (2) from the user input received,
an electrical interface (6) through which the control signal generated is transmitted to the surgical device (3), and
a mechanical assembly interface (7) which is configured to form a non-slip connection with the surgical handpiece (2) in the assembled state of the input attachment (1) in such a way that the input attachment (1) assembled with the surgical handpiece (2) can be handled as a one-piece handpiece.

2. The input attachment according to claim 1, characterized in that the assembly interface (7) comprises a receiving region (8) which is configured to form a form-fitting and/or force-fitting connection between the input attachment (1) and the surgical handpiece (2) when the input attachment (1) is placed on the surgical handpiece (2).

3. The input attachment according to claim 2, characterized in that the form-fitting and/or force-fitting connection comprises at least one of the following connections:
at least one clip for clipping the receiving region (8) to the surgical handpiece (2);
at least one magnet for magnetic coupling to an opposite pole magnet on the surgical handpiece (2);
an adhesive connection between the receiving region (8) and the surgical handpiece (2);
a bonding connection for bonding the receiving regopm (8) to the surgical handpiece (2);
a form-fitting dovetail connection or dovetail-like form-fitting connection to the surgical handpiece (2).

4. The input attachment according to claim 2, characterized in that the form-fitting and/or force-fitting connection can be released again.

5. The input attachment according to claim 2, characterized in that the receiving region (8) of the assembly interface (7) is configured to insert the surgical handpiece (2) into an inner region of the receiving region (8) and enclose it by the receiving region (8) in a non-assembled state, and to clamp the surgical handpiece (2) in the assembled state in the inner region of the receiving region (8) in a non-slip manner.

6. The input attachment according to claim 2 characterized in that the non-slip connection is formed by anti-slip elements (19) provided in the receiving region (8), which in the assembled state at least make it more difficult and in particular prevent the input attachment (1) from slipping off the surgical handpiece (2).

7. The input attachment according to claim 1, characterized in that the non-slip connection is formed by protuberances (16) provided in the receiving region (8), which in the assembled state engage in inlets of the surgical handpiece (2) and at least make it more difficult and in particular prevent the input attachment (1) from slipping off the surgical handpiece (2).

8. The input attachment according claim 1, characterized in that the electrical interface (6) comprises a bipolar electrical connection via which an input signal can be transmitted to the surgical device (3) and a response signal can be received by the surgical device (3).

9. The input attachment according to claim 1, characterized in that the electrical interface (6) is configured to couple the surgical handpiece (2) to the input attachment (1) via a direct cable connection.

10. The input attachment according to claim 1, characterized in that the electrical interface (6) is configured to couple the surgical handpiece (2) wirelessly to the input attachment (1) via an air interface.

11. The input attachment according to claim 10, characterized in that the air interface is an optical connection, in particular an infrared connection, a radio connection, for example a WLAN or mobile radio connection, and/or a Bluetooth connection.

12. The input attachment according to claim 8, characterized in that the electrical interface (6) is arranged in the region of the mechanical assembly interface (7) and is configured to be coupled, in the assembled state, to a corresponding electrical interface of the surgical handpiece (2), so as to transmit in this manner the input signal via the surgical handpiece (2) to the surgical device (3) and to receive a response signal from the surgical device (3).

13. The input attachment according to claim 1, characterized in that the input device (4) has at least one button (14), a keyboard, a touchpad, a rotary knob and/or a scroll wheel, via which user inputs can be received.

14. The input attachment according to claim 1, characterized in that the input device (4) is configured to output haptic feedback via a user input.

15. The input attachment according to claim 1, characterized in that the input device (4) and the control device (5) are configured to receive an parameter input for stimulation in the field of interoperative neuromonitoring, in particular a current intensity or a frequency, as a user input and to convert it into a corresponding control signal.

16. The input attachment according to claim 1, characterized in that the input device (4) and the control device (5) are configured to select at least one parameter of a parameter set, to receive the selected parameter as the user input and to convert it into a corresponding control signal.

17. The input attachment according to claim 1, characterized in that the control device (5) is configured to use the control signal to control at least one device-specific parameter of the surgical handpiece (2) or the surgical device (3), in particular a comment or a workflow.

18. The input attachment according to claim 1, characterized in that provision is made for a display device (24), which is configured to output a reaction signal, in particular an optical reaction signal.

19. The input attachment according to claim 8, characterized in that the display device (24) is configured to display a reaction signal received which is based on a reaction of a stimulation output by the surgical handpiece (2) and based on the user input.

20. The input attachment according to claim 18, characterized in that the display device (24) has a display and/or at least one LED for outputting the reaction signal.

21. The input attachment according to claim 18, characterized in that the display device (24) is configured to display at least one parameter selected and/or a parameter value and/or a parameter range and/or an optical warning signal.

22. The input attachment according to claim 18, characterized in that the display device (24) is configured to display at least one parameter of the surgical handpiece (2), the surgical device (3) and/or a third-party device connected to the input attachment (1).

23. The input attachment according to claim 18, characterized in that the display device (24) is designed to output a haptic feedback and/or an acoustic response signal.

24. The input attachment according to claim 18, characterized in that the display device (24) is coupled to the control device (5), via which it can be controlled.

25. A surgical device (3), comprising a first electrical device interface (10) having a first interface (10) and a second interface (11),
wherein the first interface (10) is configured to receive control signals from an input attachment (1) coupled to the surgical device (3) via the electrical device interface, and
wherein the second interface (11) is configured to transmit stimulation signals based on the control signals received to a surgical handpiece (2) coupled to the surgical device (3) via the electrical device interface (9).

26. The surgical device according to claim 25, characterized in that the surgical device (3) is configured as an interoperative neuromonitoring (IOM) device.

27. An arrangement for intraoperative use, wherein the arrangement comprises:
at least one input attachment (1), comprising an input device (4) which is configured to receive a user input;
a control device (5) which is configured to generate a control signal for controlling the surgical handpiece (2) from the user input received,
an electrical interface (6) through which the control signal generated is transmitted to the surgical device (3), and
a mechanical assembly interface (7) which is configured to form a non-slip connection with the surgical handpiece (2) in the assembled state of the input attachment (1) in such a way that the input attachment (1) assembled with the surgical handpiece (2) can be handled as a one-piece handpiece;
a surgical device (3) 26, comprising a first electrical device interface (10) having a first interface (10) and a second interface (11),
wherein the first interface (10) is configured to receive control signals from the input attachment (1) coupled to the surgical device (3) via the electrical device interface, and
wherein the second interface (11) is configured to transmit stimulation signals based on the control signals received to a surgical handpiece (2) coupled to the surgical device (3) via the electrical device interface (9); and
the surgical handpiece (2).

28. The arrangement according to claim 27, characterized in that the surgical device (3) is electrically connected to the input attachment (1) and the surgical handpiece (2) for transmitting and receiving signals, and that the surgical handpiece (2) can be mechanically fastened to the input attachment (1) in such a manner that the input attachment (1) is fastened to a position of the surgical handpiece (2) corresponding to the receiving region (8) of the input attachment (1).

29. The arrangement according to claim 27, characterized in that the surgical handpiece (3) is configured to be a monopolar or bipolar stimulation probe.

30. An arrangement according to claim 27, characterized in that the surgical handpiece (3) is configured to be a monopolar or bipolar mapping suction device.

31. (canceled)
